(19) United States
(12) Patent Application Publication
(10) Pub. No.: US 2008/0194917 A1
(43) Pub. Date: Aug. 14, 2008

(54) Monitoring Device And A Motnitoring Body-Wear

(75) Inventors: Jens Muehlsteff, Aachen (DE); Harald Reiter, Aachen (DE); Andras Montvay, Stuttgart (DE); Josef Lauter, Geilenkirchen (DE); Olaf Such, Aachen (DE); Ralf Schmidt, Aachen (DE); Michael Perkuhn, Aachen (DE); Fabian Kohler, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/595,876

(22) PCT Filed: Nov. 24, 2004
(86) PCT No.: PCT/IB2004/052522

§ 371 Date: Oct. 3, 2007

(30) Foreign Application Priority Data
Nov. 28, 2003 (EP) 03104436.5

(51) Int. Cl. A61B 5/00

(52) U.S. Cl. 600/300

(57) ABSTRACT

A monitoring device (21) arranged to measure a signal (S1) representative of a physiological condition of the individual by means of a suitably arranged sensor (22) and to automatically modify a contact pressure (P) of said sensor meeting real-time requirements. For this purpose the sensor (22) is supported by a piece of elastic material, the monitoring device further comprising an actuator (27) arranged to interact with the piece of elastic material upon receipt of a control signal (CS) from a control unit (25). The control unit (25) is arranged to analyze the signal (S1) from the sensor (22) and to decide on an alteration of the contact pressure (P). For this purpose the control means (25) comprises a logic unit (25a) arranged to carry out a suitable signal analysis. Preferably, the monitoring device (21) is arranged with a further sensor (23), a signal (S3) of which is also provided for analysis to the control unit (25).

The invention relates to a device arranged for monitoring a physiological parameter of an individual, said device comprising:

a sensor arranged to measure a signal related to said parameter when said sensor is brought into contact with the individual's skin;

an adjustable carrier arranged to support said sensor and to resiliently conform to a body part of the individual so that a contact pressure is applied to said sensor.

The invention further relates to a monitoring body-wear comprising a piece of elastic material arranged for supporting a sensor conceived to carry out a measurement of a physiological parameter of an individual when brought into contact with the individual's skin, said piece of material being arranged to conform to a body part of the individual so that a contact pressure is applied to the sensor.

An embodiment of a device as set forth in the opening paragraph is known from U.S. Pat. No. 5,353,793. The known device comprises a stretchable harness arranged with a plurality of bands whereon a set of electrodes is mountable. The known device is suited for monitoring physiological parameters of an ambulatory or a sleeping patient. In order to reduce motion artifact the known device uses stretchable bands which are placed under tension on the body of the individual and which are designed to cushion the electrodes when the patient is moving. The stretchable bands are fastened by means of a Velcro mechanism.

It is a disadvantage of the known device that the stretchable bands when fastened are worn by the individual under a constant pressure, irrespective of the activity level of the individual. If the monitoring device is conceived to be utilized for monitoring physiological parameters of the individual when he or she is exercising or actively moving, the signal quality may deteriorate substantially due to the signal artifact caused by motion yielding inaccurate measurement. Alternatively, for purposes of continuous monitoring, during a sleep phase of the individual, the contact pressure of the sensor can be decreased thus improving the individual's comfort.

It is an object of the invention to provide a monitoring device with an improved signal quality of the measured signal and an improved wearing comfort.

For this purpose the monitoring device according to the invention comprises:

an actuator arranged to interact with the adjustable carrier in order to modify the contact pressure;

control means arranged to control the contact pressure in dependence on a control signal applied to the actuator.

According to the technical measure of the invention, the monitoring device is provided with control means which adapt the contact pressure automatically thus suiting the comfort needs or signal quality requirements. The technical measure of the invention is based on the insight that there is a high correlation between a force acting on the sensor and a skin potential produced by that force. Signal quality and consequently up time of the monitoring system are strongly dependent on optimal contact pressures. The invention improves the measurement conditions for electrophysiological signals of active individuals by means of an automatic adjustment of the contact pressure of the sensor, yielding optimal signal quality as well as wearing comfort. Therefore, the same adjustable carrier can be used for all activity levels of the individual. Preferably, the contact pressure is initially selected at an intermediate level, which is then modified by the control means according to real-time requirements. It must be noted that a plurality of sensors known per se in the art is suitable for usage in the device according to the invention. For example, the sensor may comprise an ECG sensor, a respiration rate sensor, an EEG sensor, a blood-pressure sensor, a movement sensor, an activity monitor, etc.

In an embodiment of the monitoring device according to the invention the control means comprises a logic unit arranged to evaluate an objective value of the contact pressure and to determine a value of the control signal based on said evaluation. Preferably, different subject properties, like waist size, respiration range, waist shape and/or dynamics in those properties can be taken into account for evaluating the objective value of the contact pressure. For example, by assuming a cylindrical shape of a human torso, the optimal value of the contact pressure can easily be calculated by means of an appropriate geometrical model. Preferably, the adjustable carrier comprises a piece of elastic material located around a body part of the individual, so that the contact pressure can be evaluated based on a linear elasticity theory, according to:

$$p = 2 π \frac{E}{{l}_{0}} ( 1 - \frac{{l}_{0}}{u} ) ;$$

where
p is a contact pressure;
E is a modulus of elasticity of the piece of elastic material;
l₀ is a length of the piece of elastic material without strain;
u is a length of the piece of elastic material with strain.

Preferably, the logic unit comprises the calculating means arranged to carry out the determination of the objective contact pressure and a corresponding value of the control signal to be applied to the actuator. In a very simple embodiment, the calculating means is arranged to qualitatively determine the value of the control signal aiming at an increase or a decrease of the contact pressure. The optimal value of the contact pressure is obtained by means of a number of iterations.

In a further embodiment of the device according to the invention the objective value of the contact pressure is determined from a reading of a further sensor. Preferably, the further sensor is a pressure sensor located on the sensor. According to this technical measure a running value of the contact pressure can be obtained. The running value of the contact pressure is then used by the calculating means of the logic unit in order to determine the absolute value of the required length of the piece of elastic material. The relation between the required control signal and the resulting contact pressure can be pre-stored in a look-up table which is addressable by the calculating means. Additionally, other sensors can be used for determining the objective value of the contact pressure. For instance, a reading from the sensor can be made available to the control means for purposes of a suitable signal evaluation. An example of a suitable signal evaluation is a determination of the signal/noise ratio. The objective value of the contact pressure can be determined based on a signal/noise ratio. For a poor signal/noise ratio, the contact pressure must be increased. A value of a corresponding control signal to the actuator can be pre-determined and can be pre-stored in a further look-up table addressable by the calculating means. Additionally, the objective value of the contact pressure can be determined from an activity monitor, as for an active motion the objective value of the contact pressure is normally higher in order to decrease a motion artifact. A corresponding value of the control signal can be calibrated beforehand and pre-stored in a still further look-up table.

In a still further embodiment of the device according to the invention the adjustable carrier comprises a piece of elastic material, the actuator being arranged to modify the length of said piece upon receipt of the control signal thereby modifying the contact pressure. Preferably, the actuator is incorporated into the piece of elastic material. Different embodiments of a suitable actuator are envisaged, namely a piece of a polymer, a metal or a mechanical device. The principle of operation of such an actuator is based on the elasticity model. When the length of the actuator is modified the length of the piece of elastic material is changed accordingly. For instance, when the actuator is shortened by the application of a suitable control signal, the piece of elastic material is elongated and the contact pressure is increased. A memory metal incorporated into the elastic strap can change shape initiated by heat induced by a current flow and therefore can change the length of the elastic strap. A nickel-titanium alloy is a suitable example of the memory metal. Still, alternatively, a mechanical device can be used as a suitable actuator. In this case the mechanical device is arranged to change the tension of the piece of elastic material by tightening or loosening it. The elastic strap is preferably recoiled on a suitable mandrel driven by a motor.

In a still further embodiment of the monitoring device according to the invention the actuator comprises an inflatable bag, said bag being spatially arranged between the adjustable carrier and the sensor, a volume of a fluid in said bag being controlled by the control signal. According to this embodiment a localized change of the contact pressure is obtained. For situations where a high contact pressure is required this embodiment is advantageous, as the pressure on other parts of the adjustable carrier does not change, thus further improving the wearing comfort. Additionally, if the sensor comprises a plurality of sensing devices it is possible to change the contact pressure differently for different sensing devices. This technical measure enables a smart versatile monitoring device.

A monitoring body-wear according to the invention comprises an actuator arranged to interact with the piece of material in order to modify the contact pressure automatically. The monitoring body-wear according to the invention is suitable for versatile purposes, ranging from durable monitoring of a health-related parameter for medical purposes to monitoring of a vital sign of a sportsman during an exercise. Preferably, the body-wear according to the invention comprises a regular textile sewn to yield a normal piece of clothing, like a T-shirt, a brassiere or boxer pants, wherein a piece of elastic material is integrated. Preferably, the piece of elastic material is located around a waist area, thorax, or around a body extremity.

In an embodiment of the monitoring body-wear according to the invention the actuator is arranged to modify the length of said elastic material upon receipt of a control signal. The control signal is preferably provided by a suitable control means, which is preferably arranged to analyze a current value of the contact pressure or any other suitable signal, including the signal from the sensor and to determine the value of the control signal based on said analysis and on the properties of the actuator and the elastic material.

In a further embodiment of the monitoring body-wear according to the invention the actuator comprises an inflatable bag, said bag being conceived to be located between the piece of elastic material and the sensor and to change its volume upon receipt of a control signal. The control signal is preferably provided by a suitable control means, which is preferably arranged to analyze a current value of the contact pressure or any other suitable signal, including the signal from the sensor and to determine the value of the control signal based on said analysis.

These and other aspects of the invention will be discussed in further detail with reference to Figures.

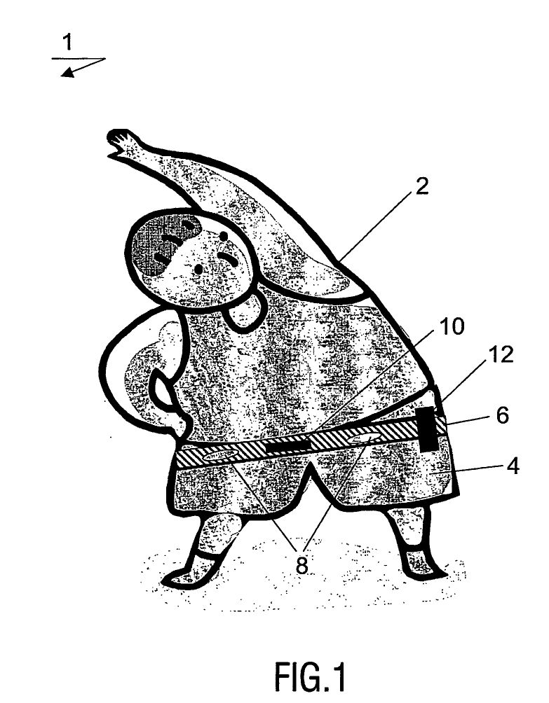

FIG. 1 presents a schematic general view of an embodiment of the monitoring device according to the invention.

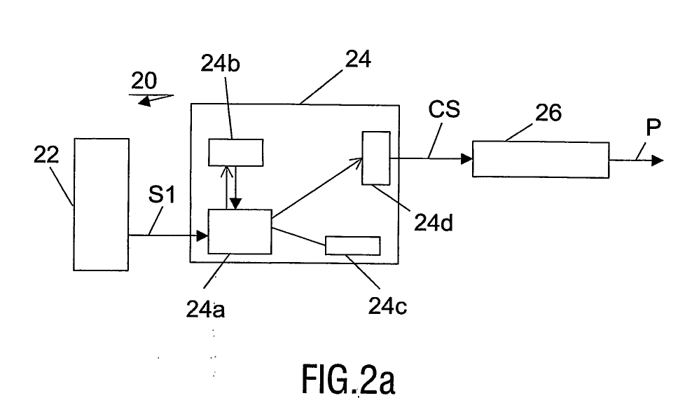

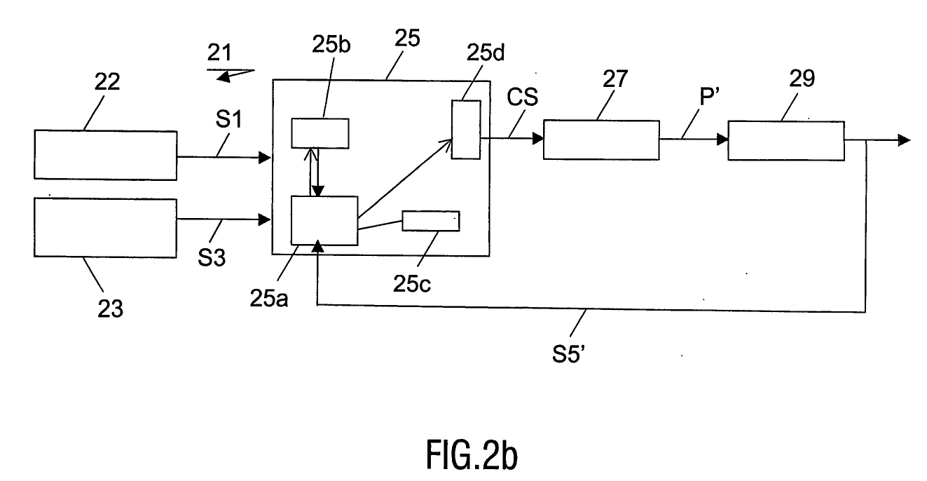

FIG. 2a presents a schematic view of a first embodiment of a block diagram of the electronics of the monitoring device.

FIG. 2b presents a schematic view of a second embodiment of a block diagram of the electronics of the monitoring device.

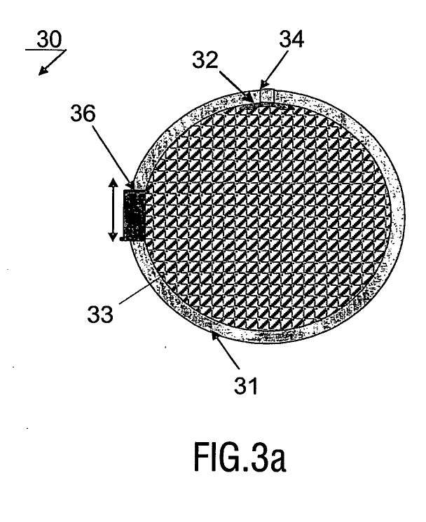

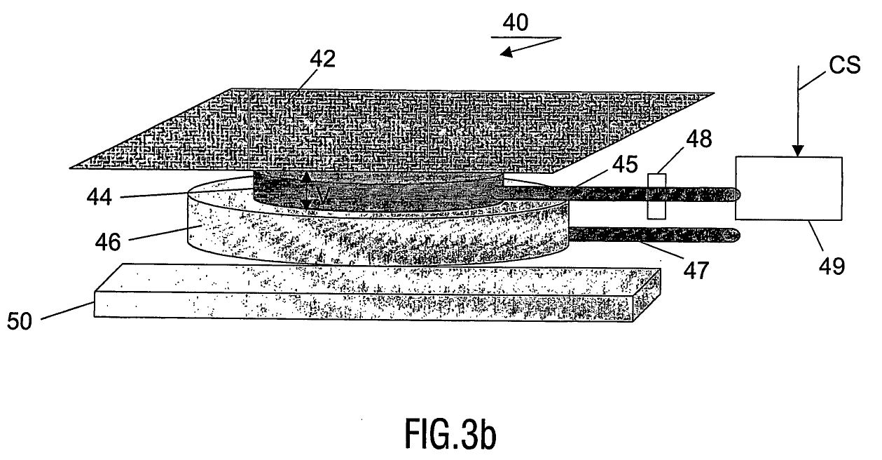

FIG. 3a presents a schematic view of a first embodiment of the actuator.

FIG. 3b presents a schematic view of a second embodiment of the actuator.

FIG. 1 presents a schematic general view of an embodiment of the monitoring device according to the invention. The individual 2 is provided with the monitoring device 1 according to the invention. The monitoring device 1 comprises an adjustable carrier 6 for housing the sensor 8. For improving the wearing comfort, the adjustable carrier 6 is integrated in a body-wear 4 comprising an elastic textile belt 6 to which a number of electrodes are attached. It must be noted that although in a current example boxer pants are depicted as a suitable adjustable carrier, any other suitably modified body-wear, like a T-shirt, a brassiere, a sock or a glove, a hat, or any other suitably arranged carrier can be used. The sensor 8 is arranged to measure a: signal representative of a physiological condition of the individual, for example an electrocardiogram, the temperature, the respiration rate, the blood pressure or any other health-related parameter. Additionally, the sensor can be arranged to determine a non-health-related parameter, like the level of a physical activity, as is known in the art of fitness. For this purpose the sensor is brought into contact with the individual's skin. Due to the elasticity of the belt 6 the sensor 8 experiences a contact pressure which keeps it substantially in place during a movement of the individual 2. The measured signal is forwarded to the control unit 12 for purposes of signal analysis and other data processing. The elastic textile belt 6 is further provided with an actuator 10 arranged to change the contact pressure of the sensor in case an activity level of the individual 2 changes. For this purpose the control unit 12 is arranged to provide a control signal (CS) to the actuator 10. Examples of the operation of the control unit and the actuator are discussed in more detail with reference to FIGS. 2a and 2b. Suitable embodiments of the adjustable carrier/actuator arrangement will be discussed with reference to FIGS. 3a and 3b.

FIG. 2a presents a schematic view of a first embodiment of a block diagram of the electronics of the monitoring device 20. The operation of the control means 24 adapted to control the contact pressure of the sensor 22 in real-time is preferably initiated by the measured signal S1, for example an ECG-pulse. The control means 24 is arranged to carry out a suitable signal analysis in order to determine whether an alteration of a current value of the contact pressure is necessary and to determine a value of the control signal to be applied to the actuator 26. For this purpose the control means 24 comprises a logic unit 24a, which is arranged to actuate calculating means 24b for purposes of signal analysis. The calculating means 24b is, for example, arranged to determine the signal to noise ratio of the input signal S1 and to report the result back to the logic unit 24a. In case a running value of the signal to noise ratio is below a predetermined threshold, the logic unit 24a addresses a look-up table 24c in order to determine the value of the control signal to be applied to the actuator by the control signal delivery means 24d. When the value of the control signal is determined, the logic unit 24a discharges a trigger signal to the control signal delivery means 24d. Upon receipt of the control signal (not shown) by the actuator 26 the control pressure P is altered accordingly.

FIG. 2b presents a schematic view of a second embodiment of a block diagram of the electronics of the monitoring device 21. The control means 25 is arranged to receive a signal S1 from the sensor 22 together with a further signal S3 from a further sensor 23. An example of a suitable further sensor is an activity sensor, a pressure sensor, a strain sensor, a timer, a temperature sensor, etc. Inclusion of a further sensor into a decision making circuit is advantageous, as it provides further information, like the level of a user activity (activity sensor), the degree of tightness of the elastic material (strain sensor integrated into the elastic material), current ambient time (timer), etc. Preferably, the further signal S3 is analyzed by the logic unit 25a together with the measured signal S1 and a decision on an alteration of the contact pressure is taken based on this analysis. Preferably, also a value of the control signal is determined from functional dependencies between these signals, said dependencies being analyzed by the calculating means 25b. Optionally, a value of the control signal is stored in a look-up table 25c. Upon receipt of a suitable trigger signal from the control unit 25a by the control signal delivery means 25d, the control signal CS is applied to the actuator 27. The actuator interacts with the adjustable carrier and the contact pressure P is modified accordingly. Preferably, the adjustable carrier is supplied with a pressure sensor 29 arranged on the sensor or in the direct vicinity thereof. The pressure sensor 29 provides a reference signal S5′ to the control means for verification purposes. The current embodiment of the electronics of the monitoring device 21 is arranged with a control loop which enables reliable operation of the pressure control system, said system being accommodatable to different activity levels of the individual.

FIG. 3a presents a schematic view of a first embodiment of the actuator. The monitoring device 30 is worn by an individual so that a relevant cross-section 33 is surrounded by the elastic material 31. The length of the elastic material is selected so that the sensor 32 experiences a contact pressure when put into contact with the individual's skin. In order to determine an absolute value of the contact pressure a suitable pressure sensor 34 is arranged on top of the sensor 32. An actuator 36 is attached to the elastic material for purposes of contact pressure control. Preferably, the actuator comprises a piece of a resilient material, like a polymer, or a mechanical device which changes its length 38 in response to the control signal (not shown).

FIG. 3b presents a schematic view of a second embodiment of the actuator. The monitoring device 40 comprises an adjustable carrier 42, a sensor 46 and an inflatable bag 44. The inflatable bag 44 can be inflated by liquid or air from a corresponding pump 49 by means of a duct 45. The adjustable carrier 42 is selected in such a way that the sensor 46 experiences a contact pressure when put into contact with an individual's skin 50. The measured signal from the sensor 46 is made available to a control unit (not shown) by means of a suitable wiring 47. In case it is determined that the signal quality deteriorates, the control means delivers a control signal CS to the pump 49 so that a volume V of the inflatable bag is modified yielding a required contact pressure. Additionally, a pressure sensor 48 can be arranged in the duct 45 to yield a direct measure of the induced contact pressure.

Although the invention has been described with reference to preferred embodiments thereof, it is to be understood that these are not limitative examples. Thus, various modifications may be apparent to those skilled in the art, without departing from the scope of the invention, as defined by the claims. The invention can be implemented by means of both hardware and software, and several ‘means’ may be represented by the same item in hardware. Any reference signs do not limit the scope of the claims.

1. A device (1) arranged for monitoring a physiological parameter of an individual (2), said device comprising:
a sensor (8) arranged to measure a signal (S1) related to said parameter when said sensor is brought into contact with the individual's skin (50);
an adjustable carrier (6) arranged to support said sensor and to resiliently conform to a body part of the individual so that a contact pressure is applied to the sensor (8);
an actuator (10) arranged to interact with the adjustable carrier (6) in order to modify the contact pressure;
control means (12) arranged to control the contact pressure in dependence on a control signal applied to the actuator (10).

2. A device according to claim 1, wherein the control means (24,25) comprises a logic unit (24a, 25a) arranged to evaluate an objective value of the contact pressure (P) and to determine a value of the control signal (CS) based on said evaluation.

3. A device according to claim 2, wherein the objective value of the contact pressure (P) is determined from a reading of a further sensor (23).

4. A device according to claim 2, wherein the adjustable carrier (31) comprises a piece of elastic material, the actuator (36) being arranged to modify the length of said piece upon receipt of the control signal (CS) thereby modifying the contact pressure.

5. A device according to claim 2, wherein the actuator comprises an inflatable bag (46), said bag being spatially arranged between the adjustable carrier (42) and the sensor (46), a volume of a fluid in said bag being controlled by the control signal (CS).

6. A monitoring body-wear (4) comprising:
a piece of elastic material (6) arranged for supporting a sensor (8) conceived to carry out a measurement of a physiological parameter (S1) of an individual when brought into contact with the individual's skin, said piece of material being arranged to conform to a body part of the individual so that a contact pressure (P) is applied to the sensor (8), said monitoring body-wear further comprising:
an actuator (10) arranged to interact with the piece of material (6) in order to modify the contact pressure (P) automatically.

7. A body-wear according to claim 6, wherein the actuator (36) is arranged to modify the length of said elastic material (31) upon receipt of a control signal (CS).

8. A body-wear according to claim 6, wherein the actuator comprises an inflatable bag (46), said bag being conceived to be located between the piece of elastic material (42) and the sensor (46) and to change it volume upon receipt of a control signal (CS).
